(19) United States
(12) Patent Application Publication
(10) Pub. No.: US 2004/0128709 A1
(43) Pub. Date: Jul. 1, 2004

(54) Composite utilization of a group of genes in biosynthetic pathway of caffeine

(76) Inventors: Hirotaka Uefuji, Nara-shi (JP); Hiroshi Sano, Ikoma-shi (JP); Nozomu Koizumi, Ikoma-shi (JP); Atsuhiko Shinmyo, Ikoma-gun (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/623,854

(22) Filed: Jul. 22, 2003

(30) Foreign Application Priority Data
Jul. 23, 2002 (JP) 2002-213655

(51) Int. Cl. C12P 17/16; C07D 473/02; A01H 1/00; C12N 15/82

(52) U.S. Cl. 800/278; 435/118; 544/267

(57) ABSTRACT

In the biosynthetic reaction system of caffeine, enzymes which catalyze these reactions respectively, and a method of composite utilization of genes encoding these enzymes, respectively are provided. A process for producing 7-methylxanthine, theobromine or caffeine which comprises methylation of xanthosine, ribose removal of 7-methylxanthosine, methylation of 7-methylxanthine, and/or methylation of theobromine ex vivo, in the presence of a combination of two or more of the enzymes a, c and d, and the cellular extract b. a: an enzyme which catalyzes methylation of xanthosine at the 7-position of the purine ring and has the amino acid sequence set out in SEQ ID NO: 1, b: a crude cellular extract obtained from Escherichia coli which catalyzes ribose removal of 7-methylxanthosine at the 9-position of the purine ring, c: an enzyme which catalyzes methylation of 7-methylxanthine at the 3-position of the purine ring and has the amino acid sequence set out in SEQ ID NO: 4, d: an enzyme which catalyzes methylation of theobromine at the 1-position of the purine ring and has the amino acid sequence set out in SEQ ID NO: 7.

### BACKGROUND OF THE INVENTION

1. Field of the Invention

The present invention relates to xanthosine methyltransferase, 7-methylxanthine methyltransferase (theobromine synthase) and 3,7-dimethylxanthine methyltransferase (caffeine synthase) which catalyze respective steps in a series of a reaction system in which caffeine is synthesized from xanthosine via7-methylxanthosine, 7-methylxanthine and theobromine, and a method of composite utilization of multiple genes in the biosynthetic pathway of caffeine encoding these enzymes, i.e., a method in which two or more of the multiple enzymes described above are used in combination and a method in which two or more of the multiple genes in the biosynthetic pathway of caffeine described above are used in combination.

2. Description of the Related Art

Caffeine is a purine alkaloid which is biologically synthesized in plant species such as coffee (Coffea), tea (Camellia sinensis), cola (Cola acuminate) and mate (Ilex paraguariensis). Caffeine has been widely utilized as a medicine because it has a variety of pharmacological actions such as central nervous system stimulatory action S. Further, potential ability of this compound capable of being utilized as a pesticide is suggested because it has feeding-repellent effects and pesticidal effects against insects and the like.

Caffeine is currently produced through extracting from the aforementioned plants or through chemical synthesis. Accordingly, development of techniques has been desired in which microorganisms or plants which do not biologically synthesize caffeine in themselves are allowed to produce caffeine through utilizing a gene recombination technique for the purpose of supplying caffeine on a large scale at a lower cost. In addition, such techniques to allow production of caffeine in plants are also expected as a process for directly defending the plant from pest feeding by a herbivore.

In coffee plants or tea plants, caffeine is biologically synthesized from xanthosine as a starting material, which is an intermediate catabolic product of adenine nucleotide and guanine nucleotide, via 7-methylxanthosine, 7-methylxanthine and theobromine (FIG. 1). A series of this reaction is catalyzed by xanthosine methyltransferase, an enzyme to remove the ribose moiety of 7-methylxanthosine, theobromine synthase and caffeine synthase. CaMXMT cDNA encoding the theobromine synthase was already isolated from coffee plants (see, Ogawa et al., J. Biol. Chem., 276, 8213-8218 (2001). Moreover, JP-A No. 2001-37490 describes the amino acid sequence of methyltransferase which catalyzes two steps of methylation reactions: 7-methylxanthine→theobromine→caffeine, and the nucleotide sequence encoding the same. Further, this gazette describes a process for modifying the constitution of methylxanthines in a plant which biologically synthesizes the methylxanthines (7-methylxanthine, paraxanthine, theobromine and caffeine) through introducing a DNA having the nucleotide sequence. However, any technique has not been known heretofore to allow this compound to be produced in a living organism species which can not biologically synthesize methylxanthines in itself.

### SUMMARY OF THE INVENTION

An object of the present invention is to provide enzymes respectively catalyze the reactions in a series of the reaction to produce caffeine from xanthosine via 7-methylxanthosine, 7-methylxanthine and theobromine, and a method of composite utilization of genes encoding these enzymes respectively. For example, following objects can be accomplished by the present invention. (1) To produce methylxanthines (7-methylxanthine, theobromine or caffeine) from xanthosine using two or more of the multiple enzymes in combination which catalyze each step in the series of the aforementioned caffeine biosynthetic reaction system. (2) To alter the metabolism of an organism which does not biologically synthesize methylxanthines in itself to allow for production of these compounds. (3) To defend a plant from pest feeding by a herbivore by the process (2) described above.

The present inventors elaborately investigated to solve the aforementioned problems, and obtained the following findings. First, DNA fragments having each of the nucleotide sequence set out in SEQ ID NO: 2, 5 and 8 in Sequence Listing were amplified by PCR. Next, these DNA fragments were incorporated into an expression vector, and the resulting recombinant vector was introduced into Escherichia coli to express a large amount of the recombinant protein derived from the DNA. After crude extraction or purification of this recombinant protein, enzymological characteristics thereof were studied. Accordingly, it was found that the protein derived from the nucleotide sequence set out in SEQ ID NO: 2 catalyzed a productive reaction of 7-methylxanthosine through methylation of xanthosine; that the protein derived from the nucleotide sequence set out in SEQ ID NO: 5 catalyzed a productive reaction of theobromine through methylation of 7-methylxanthine; and that the protein derived from the nucleotide sequence set out in SEQ ID NO: 8 catalyzed a productive reaction of caffeine through methylation of theobromine. In other words, it was confirmed that the DNAs of this group encode xanthosine methyltransferase, theobromine synthase and caffeine synthase, respectively. In addition, it was also found that the cellular extract of Escherichia coli catalyzed a productive reaction of 7-methylxanthine through ribose removal of 7-methylxanthosine. More specifically, it was found that the cellular extract of Escherichia coli which do not biologically synthesize methylxanthines in themselves also has an activity of an enzyme to remove the ribose moiety of 7-methylxanthosine (7-methylxanthosine hydrolase or 7-methylxanthosine phosphorolytic enzyme). Moreover, a mixture of the aforementioned three recombinant proteins and the cellular extract of Escherichia coli catalyzes a productive reaction from xanthosine to caffeine. In other words, it was confirmed that using the activities of the three methyltransferases and the activity of the one enzyme to remove the ribose moiety, a biosynthetic pathway of caffeine can be reconstituted in an ex vivo, i.e., cell free system, in other words, the pathway can be artificially constituted.

As the enzymes and cellular extract used in the reconstitution of the biosynthetic pathway of caffeine, those derived from any organism can be applied as long as they have comparable activities thereto. For example, instead of the theobromine synthase and caffeine synthase derived from coffee plants, methyltransferase derived from tea plants can be utilized, which catalyzes two steps of methylation reactions: 7-methylxanthine theobromine caffeine, as described in JP-A No. 2001-37490.

Moreover, reconstitution of the biosynthetic pathway of caffeine can be performed not only in a cell free system using the aforementioned enzymes and cellular extract, but also in a transformed biological system produced by introducing genes encoding these enzymes into any of organism species. Xanthosine which may become a starting material of biosynthesis of caffeine is present as an intermediate product of purine nucleotide catabolism in a wide range of organism species in addition to coffee plants and tea plants. Further, the enzyme to remove the ribose moiety of a purine nucleoside is known to react not only toward specific purine nucleoside derivatives but also toward various purine nucleoside derivatives, in general (see, Guranowski, Plant Physiol., 70, 344-349(1982); Parkin, J. Biol. Chem., 271, 21713-21719 (1996); Ogawa et al., Appl. Environ. Microbiol., 67, 1783-1787(2001)), and thus it is believed that the reaction of ribose removal of 7-methylxanthosine may be catalyzed by an enzyme to remove the ribose moiety of purine nucleoside, which is carried by general organisms. This can be readily expected also from the finding that Escherichia coli which do not biologically synthesize methylxanthines in themselves have an activity to remove the ribose moiety of 7-methylxanthosine (see, Examples herein). In brief, a biosynthetic pathway of caffeine can be reconstituted in wide variety of organism species through introducing and allowing the expression of a xanthosine methyltransferase gene, a theobromine synthase gene and a caffeine synthase gene.

The present invention was accomplished on the basis of the findings hereinabove.

### BRIEF DESCRIPTION OF THE DRAWINGS

FIG. 1 is a flow sheet illustrating a primary biosynthetic pathway of caffeine in coffee plants and tea plants. Name of each compound is described beneath each formula. SAM means S-adenosyl-L-methionine, and SAH means S-adenosyl-L-homocysteine. The reaction represented by (1) is catalyzed by xanthosine methyltransferase; the reaction represented by (2) is catalyzed by an enzyme to remove the ribose moiety of 7-methylxanthosine; the reaction represented by (3) is catalyzed by 7-methylxanthine methyltransferase (theobromine synthase); and the reaction represented by (4) is catalyzed by 3,7-dimethylxanthine methyltransferase (caffeine synthase).

FIG. 2 is a drawing schematically illustrating pGEX-CaMTL3 vector, pGEX-CaMTL4 vector and pGEX-CaMTL5 vector. These vectors are those constructed by ligating CaMTL3 cDNA (SEQ ID NO: 2), CaMTL4 cDNA (SEQ ID NO: 5) or CaMTL5 cDNA (SEQ ID NO: 8), which is a predicted methyltransferase gene (MTL), downstream of a glutathione S-transferase gene (GST) controlled by tac promoter (Ptac). Ampicillin resistant gene (Amp^{r}), lac repressorgene (lacI^{q}) and pBR322 replication origin (pBR322ori) are shown in the Figure.

FIG. 3 illustrates detection of recombinant proteins by the SDS-PAGE analysis. After separating crude extract sample of GST, crude extract sample of a fusion protein of GST and CaMTL3 (GST-MTL3, shown by crude extract sample MTL3 in FIG. 3), purified sample of GST, purified sample of a fusion protein of GST and CaMTL3 (GST-MTL3, shown by purified sample MTL3 in FIG. 3), purified sample of a fusion protein of GST and CaMTL4 (GST-MTL4, shown by purified sample MTL4 in FIG. 3), and purified sample of a fusion protein of GST and CaMTL5 (GST-MTL5, shown by purified sample MTL5 in FIG. 3) on a 9% gel, proteins were detected by Coomassie brilliant blue staining. Asterisks denote bands of the recombinant proteins.

FIG. 4 illustrates detection by the thin layer chromatography (TLC) analysis of products of the enzymatic reactions. Enzymatic reactions were performed using the recombinant protein samples shown in FIG. 3. Left panel illustrates states of detection by auto radiography of spots of products of the enzymatic reactions which were obtained by adding a methyl group (¹⁴C-labelled) after developing the enzymatic reaction mixture. Methyl group acceptors used in the enzymatic reactions are shown above the left panel, and specific products of the enzymatic reactions are shown beneath the left panel. Right panel illustrates states of detection by UV illumination of spots of standards after the standards were developed. Left and right panels were from one TLC plate which was divided in two parts after development. Starting points and the mobility are shown between the left and right panels. XR means xanthosine; 7mXR means 7-methylxanthosine; 7mX means 7-methylxanthine; and SAM means S-adenosyl-L-methionine.

FIG. 5 illustrates detection by the TLC analysis of products of the enzymatic reactions. Enzymatic reactions were performed using the recombinant protein samples shown in FIG. 3. Left panel illustrates states of detection by auto radiography of spots of products of the enzymatic reactions which were obtained by adding a methyl group (¹⁴C-labelled) after developing the enzymatic reaction mixture. Methyl group acceptors used in the enzymatic reactions are shown above the left panel, and specific products of the enzymatic reactions are shown beneath the left panel. Right panel illustrates states of detection by UV illumination of spots of standards after the standards were developed. Left and right panels were from one TLC plate which was divided in two parts after development. Starting points and the mobility are shown between the left and right panels. 7mX means 7-methylxanthine; Tb means theobromine; Px means paraxanthine; Cf means caffeine; and SAM means S-adenosyl-L-methionine.

FIG. 6 illustrates detection by the TLC analysis of products of enzymatic reactions. Enzymatic reactions were performed using the recombinant protein samples shown in FIG. 3. Left panel illustrates states of detection by auto radiography of spots of products of the enzymatic reactions which were obtained by adding a methyl group (¹⁴C-labelled) after developing the enzymatic reaction mixture or the purified product (lanes indicated by the asterisks). Methyl group acceptors used in the enzymatic reactions are shown above the left panel, and specific products of the enzymatic reactions are shown beneath the left panel. Right panel illustrates states of detection by UV illumination of spots of standards after the standards were developed. Left and right panels were from one TLC plate which was divided in two parts after development. Starting points and the mobility are shown between the left and right panels. 7mX means 7-methylxanthine; Tb means theobromine; Px means paraxanthine; Cf means caffeine; and SAM means S-adenosyl-L-methionine.

FIG. 7 illustrates detection of products of enzymatic reactions by an HPLC analysis. Enzymatic reactions were performed using the recombinant protein samples shown in FIG. 3, and xanthosine alone as a methyl receptor. Chromatograms of the reaction mixtures are shown which were performed using: (A) crude extract MTL3 alone, (B) crude extract MTL3 and purified MTL4, and (C) crude extract MTL3, purified MTL4 and purified MTL5. (D) shows a chromatogram of the standards. Closed arrow heads indicate peaks of specific products and the standard. Open arrowheads indicate peaks of impurities. 7mX means 7-methylxanthine; Tb means theobromine; and Cf means caffeine.

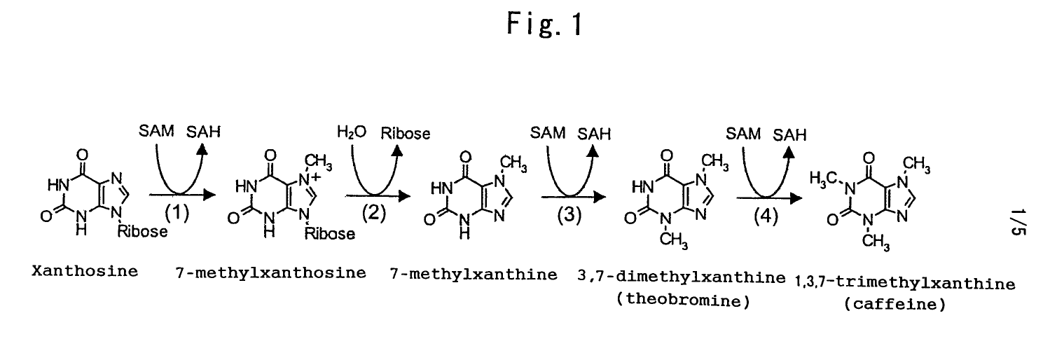

### DETAILED DESCRIPTION OF THE INVENTION

The present invention is explained in detail below.

An aspect of the invention (first invention) is to provide a process for producing 7-methylxanthine, theobromine or caffeine which comprises: methylation of xanthosine at the 7-position of the purine ring; ribose removal of 7-methlxanthosine at the 9-position of the purine ring; methylation of 7-methylxanthine at the 3-position of the purine ring; and/or methylation of theobromine at the 1-position of the purine ring ex viva, under the catalytic action of a combination of two or more of the following enzymes (a), (c) and (d), and the cellular extract (b):

(a) an enzyme having a catalytic activity of methylation of xanthosine at the 7-position of the purine ring and having the amino acid sequence set out in SEQ ID NO: 1,

(b) a crude cellular extract obtained from Escherichia coli having a catalytic activity of ribose removal of 7-methlxanthosine at the 9-position of the purine ring,

(c) an enzyme having a catalytic activity of methylation of 7-methylxanthine at the 3-position of the purine ring and having the amino acid sequence set out in SEQ ID NO: 4,

(d) an enzyme having a catalytic activity of methylation of theobromine at the 1-position of the purine ring and having the amino acid sequence set out in SEQ ID NO: 7.

In this first invention, at least one of the enzymes (a), (c) and (d), and the cellular extract (b) may be substituted for one having a comparable activity thereto. Next, second to eighth inventions relating to a process for reconstituting a biosynthetic pathway of caffeine in a transformed organism system according to the invention are explained.

Another aspect of the invention (second invention) relates to a process for producing theobromine or caffeine which comprises allowing the expression of a combination of two or more of the following DNA molecules (a), (b) and (c) in a host organism to alter the metabolism of the host organism. More particularly, the second invention is a process for producing theobromine or caffeine which comprises allowing the expression of a combination of (a)+(b) or a combination of (a)+(b)+(c) of the following DNA molecules (a), (b) and (c) in a host organism which biologically synthesizes xanthosine and has an enzymatic activity of ribose removal of 7-methylxanthosine to alter the metabolism of the host organism:

(a) a DNA molecule encoding an enzyme which catalyzes methylation of xanthosine at the 7-position of the purine ring and having the nucleotide sequence set out in SEQ ID NO: 2,

(b) a DNA molecule encoding an enzyme which catalyzes methylation of 7-methylxanthine at the 3-position of the purine ring and having the nucleotide sequence set out in SEQ ID NO: 5,

(c) a DNA molecule encoding an enzyme which catalyzes methylation of theobromine at the 1-position of the purine ring and having the nucleotide sequence set out in SEQ ID NO: 8.

Yet another aspect of the invention (third invention) is to provide a process for producing theobromine or caffeine which comprises allowing the expression of a combination of the following DNA molecules (b) and (c) in a host organism which biologically synthesizes 7-methylxanthine to alter the metabolism of the host organism:

(b) a DNA molecule encoding an enzyme which catalyzes methylation of 7-methylxanthine at the 3-position of the purine ring and having the nucleotide sequence set out in SEQ ID NO: 5,

(c) a DNA molecule encoding an enzyme which catalyzes methylation of theobromine at the 1-position of the purine ring and having the nucleotide sequence set out in SEQ ID NO: 8.

Still another aspect of the invention (fourth invention) is to provide a process for altering the amount of production of theobromine or caffeine which comprises allowing the expression of a combination of two or more of the following DNA molecules (a), (b) and (c) in a host organism which biologically synthesizes theobromine or caffeine:

(a) a DNA molecule encoding an enzyme which catalyzes methylation of xanthosine at the 7-position of the purine ring and having the nucleotide sequence set out in SEQ ID NO:2,

a DNA molecule encoding an enzyme which catalyzes methylation of 7-methylxanthine at the 3-position of the purine ring and having the nucleotide sequence set out in SEQ ID NO: 5,

(b) a DNA molecule encoding an enzyme which catalyzes methylation of theobromine at the 1-position of the purine ring and having the nucleotide sequence set out in SEQ ID NO: 8.

In the second to fourth inventions, at least one of the DNA molecules (a), (b) and (c) may be substituted for one having a comparable function thereto.

Further aspect of the invention (fifth invention) relates to a process for producing theobromine or caffeine which comprises allowing the expression of a combination of two or more of the following RNA molecules (a), (b) and (c) in a host organism to alter the metabolism of the host organism. More particularly, the fifth invention is a process for producing theobromine or caffeine which comprises allowing the expression of a combination of (a)+(b) or a combination of (a)+(b)+(c) of the following RNA molecules (a), (b) and (c) in a host organism which biologically synthesizes xanthosine and has an enzymatic activity of ribose removal of 7-methylxanthosine to alter the metabolism of the host organism:

(a) an RNA molecule encoding an enzyme which catalyzes methylation of xanthosine at the 7-position of the purine ring and having the nucleotide sequence set out in SEQ ID NO: 3,

(b) an RNA molecule encoding an enzyme which catalyzes methylation of 7-methylxanthine at the 3-position of the purine ring and having the nucleotide sequence set out in SEQ ID NO: 6,

(c) an RNA molecule encoding an enzyme which catalyzes methylation of theobromine at the 1-position of the purine ring and having the nucleotide sequence set out in SEQ ID NO: 9.

Still another aspect of the invention (sixth invention) is to provide a process for producing theobromine or caffeine which comprises allowing the expression of a combination of the following RNA molecules (b) and (c) in a host organism which biologically synthesizes 7-methylxanthine to alter the metabolism of the host organism:

(b) an RNA molecule encoding an enzyme which catalyzes methylation of 7-methylxanthine at the 3-position of the purine ring and having the nucleotide sequence set out in SEQ ID NO: 6,

(c) an RNA molecule encoding an enzyme which catalyzes methylation of theobromine at the 1-position of the purine ring and having the nucleotide sequence set out in SEQ ID NO: 9.

Yet another aspect of the invention (seventh invention) is to provide a process for altering the amount of production of theobromine or caffeine which comprises allowing the expression of a combination of two or more of the following RNA molecules (a), (b) and (c) in a host organism which biologically synthesizes theobromine or caffeine:

(a) an RNA molecule encoding an enzyme which catalyzes methylation of xanthosine at the 7-position of the purine ring and having the nucleotide sequence set out in SEQ ID NO:3,

(b) an RNA molecule encoding an enzyme which catalyzes methylation of 7-methylxanthine at the 3-position of the purine ring and having the nucleotide sequence set out in SEQ ID NO: 6,

(c) an RNA molecule encoding an enzyme which catalyzes methylation of theobromine at the 1-position of the purine ring and having the nucleotide sequence set out in SEQ ID NO: 9.

In the fifth to seventh inventions, at least one of the RNA molecules (a), (b) and (c) may be substituted for one having a comparable function thereto.

Further aspect of the invention (eighth invention) is to provide a process according to any one of the second to seventh inventions wherein the host organism is a plant, and production or increase of production of theobromine or caffeine defends the host plant from pest feeding by a herbivore.

### DESCRIPTION OF THE PREFERRED EMBODIMENTS

The first invention relates to a process for reconstituting a biosynthetic pathway of caffeine in a cell free system, which is a process for producing 7-methylxanthine, theobromine or caffeine which comprises methylation of xanthosine at the 7-position of the purine ring, ribose removal of 7-methylxanthosine at the 9-position of the purine ring, methylation of 7-methylxanthine at the 3-position of the purine ring, and/or methylation of theobromine at the 1-position of the purine ring ex vivo, under the catalytic action of a combination of two or more of the following enzymes (a), (c) and (d), and the cellular extract (b):

(a) an enzyme having a catalytic activity of methylation of xanthosine at the 7-position of the purine ring and having the amino acid sequence set out in SEQ ID NO: 1,

(b) a crude cellular extract obtained from Escherichia coli having a catalytic activity of ribose removal of 7-methlxanthosine at the 9-position of the purine ring,

(c) an enzyme having a catalytic activity of methylation of 7-methylxanthine at the 3-position of the purine ring and having the amino acid sequence set out in SEQ ID NO: 4,

(d) an enzyme having a catalytic activity of methylation of theobromine at the 1-position of the purine ring and having the amino acid sequence set out in SEQ ID NO: 7.

Xanthosine methyltransferase which catalyzes methylation of xanthosine, theobromine synthase which catalyzes methylation of 7-methylxanthine, and caffeine synthase which catalyzes methylation of theobromine are generically referred to herein as caffeine synthetic methyltransferase.

In the first invention, at least one of the enzymes (a), (c) and (d), and the cellular extract (b) may be substituted for one having a comparable activity thereto. For example, instead of the theobromine synthase and caffeine synthase derived from coffee plants, methyltransferase derived from tea plants can be utilized, which catalyzes two steps of methylation reactions: 7-methylxanthine→theobromine→caffeine, as described in JP-A No. 2001-37490.

The aforementioned (a) and ones comparable thereto are generically referred to as xanthosine methyltransferase, the aforementioned (b) and ones comparable thereto are generically referred to as the enzyme to remove the ribose moiety of 7-methylxanthosine, the aforementioned (c) and ones comparable thereto are generically referred to as theobromine synthase, and the aforementioned (d) and ones comparable thereto are generically referred to as caffeine synthase herein, respectively.

Examples of the combination of the enzyme and cellular extract include a combination of (a)+(b)+(c)+(d) for synthesizing caffeine from xanthosine, a combination of (a)+(b)+(c) for synthesizing theobromine from xanthosine, and a combination of (c)+(d) for synthesizing caffeine from 7-methylxanthine.

The second invention which relates to a process for reconstituting a biosynthetic pathway of caffeine in a transformed organism system according to the invention is a process for producing theobromine or caffeine which comprises allowing the expression of a combination of (a)+(b) or a combination of (a)+(b)+(c) of the following DNA molecules (a), (b) and (c) in a host organism which biologically synthesizes xanthosine and has an enzymatic activity of ribose removal of 7-methylxanthosine to alter the metabolism of the host organism:

(a) a DNA molecule encoding an enzyme which catalyzes methylation of xanthosine at the 7-position of the purine ring and having the nucleotide sequence set out in SEQ ID NO: 2,

(b) a DNA molecule encoding an enzyme which catalyzes methylation of 7-methylxanthine at the 3-position of the purine ring and having the nucleotide sequence set out in SEQ ID NO: 5,

(c) a DNA molecule encoding an enzyme which catalyzes methylation of theobromine at the 1-position of the purine ring and having the nucleotide sequence set out in SEQ ID NO: 8.

In the second invention, a combination of (a)+(b)+(c) is used for synthesizing caffeine from xanthosine, and a combination of (a)+(b) is used for synthesizing theobromine from xanthosine.

The third invention is a process for producing theobromine or caffeine which comprises allowing the expression of a combination of the following DNA molecules (b) and (c) in a host organism which biologically synthesizes 7-methylxanthine to alter the metabolism of the host organism:

(b) a DNA molecule encoding an enzyme which catalyzes methylation of 7-methylxanthine at the 3-position of the purine ring and having the nucleotide sequence set out in SEQ ID NO: 5,

(c) a DNA molecule encoding an enzyme which catalyzes methylation of theobromine at the 1-position of the purine ring and having the nucleotide sequence set out in SEQ ID NO: 8.

The fourth invention is a process for altering the amount of production of theobromine or caffeine which comprises allowing the expression of a combination of two or more of the following DNA molecules (a), (b) and (c) in a host organism which biologically synthesizes theobromine or caffeine:

(a) a DNA molecule encoding an enzyme which catalyzes methylation of xanthosine at the 7-position of the purine ring and having the nucleotide sequence set out in SEQ ID NO:2,

(b) a DNA molecule encoding an enzyme which catalyzes methylation of 7-methylxanthine at the 3-position of the purine ring and having the nucleotide sequence set out in SEQ ID NO: 5,

(c) a DNA molecule encoding an enzyme which catalyzes methylation of theobromine at the 1-position of the purine ring and having the nucleotide sequence set out in SEQ ID NO: 8.

In the second to fourth inventions, at least one of the DNA molecules (a), (b) and (c) may be substituted for one having a comparable function thereto.

In the second to fourth inventions, 7-methylxanthine, theobromine or caffeine is obtained through methylation of xanthosine at the 7-position of the purine ring, ribose removal of 7-methylxanthosine at the 9-position of the purine ring, methylation of 7-methylxanthine at the 3-position of the purine ring, and/or methylation of theobromine at the 1-position of the purine ring.

The fifth invention is a process for producing theobromine or caffeine which comprises allowing the expression of a combination of (a)+(b) or a combination of (a)+(b)+(c) of the following RNA molecules (a), (b) and (c) in a host organism which biologically synthesizes xanthosine and has an enzymatic activity of ribose removal of 7-methylxanthosine to alter the metabolism of the host organism:

(a) an RNA molecule encoding an enzyme which catalyzes methylation of xanthosine at the 7-position of the purine ring and having the nucleotide sequence set out in SEQ ID NO: 3,

(b) an RNA molecule encoding an enzyme which catalyzes methylation of 7-methylxanthine at the 3-position of the purine ring and having the nucleotide sequence set out in SEQ ID NO: 6,

(c) an RNA molecule encoding an enzyme which catalyzes methylation of theobromine at the 1-position of the purine ring and having the nucleotide sequence set out in SEQ ID NO: 9.

In the fifth invention, a combination of (a)+(b)+(c) is used for synthesizing caffeine from xanthosine, and a combination of (a)+(b) is used for synthesizing theobromine from xanthosine.

The sixth invention is a process for producing theobromine or caffeine which comprises allowing the expression of a combination of the following RNA molecules (b) and (c) in a host organism which biologically synthesizes 7 -methylxanthine to alter the metabolism of the host organism:

(b) an RNA molecule encoding an enzyme which catalyzes methylation of 7-methylxanthine at the 3-position of the purine ring and having the nucleotide sequence set out in SEQ ID NO: 6,

(c) an RNA molecule encoding an enzyme which catalyzes methylation of theobromine at the 1-position of the purine ring and having the nucleotide sequence set out in SEQ ID NO: 9.

The seventh invention is a process for altering the amount of production of theobromine or caffeine which comprises allowing the expression of a combination of two or more of the following RNA molecules (a), (b) and (c) in a host organism which biologically synthesizes theobromine or caffeine:

(a) an RNA molecule encoding an enzyme which catalyzes methylation of xanthosine at the 7-position of the purine ring and having the nucleotide sequence set out in SEQ ID NO:3,

(b) an RNA molecule encoding an enzyme which catalyzes methylation of 7-methylxanthine at the 3-position of the purine ring and having the nucleotide sequence set out in SEQ ID NO: 6,

(c) an RNA molecule encoding an enzyme which catalyzes methylation of theobromine at the 1-position of the purine ring and having the nucleotide sequence set out in SEQ ID NO: 9.

In the fifth to seventh inventions, at least one of the RNA molecules (a), (b) and (c) may be substituted for one having a comparable function thereto.

In the fifth to seventh inventions, 7-methylxanthine, theobromine or caffeine is obtained through methylation of xanthosine at the 7-position of the purine ring, ribose removal of 7-methylxanthosine at the 9-position of the purine ring, methylation of 7-methylxanthine at the 3-position of the purine ring, and/or methylation of theobromine at the 1-position of the purine ring.

The eighth aspect of the invention is a process according to any one of second to seventh inventions wherein the host organism is a plant, and production or increase of production of theobromine or caffeine defends the host plant from pest feeding by a herbivore.

The DNA molecules encoding the aforementioned caffeine biosynthetic methyltransferases can be isolated from an organism which produces the caffeine biosynthetic methyltransferase utilizing for example, a PCR technique in which an origo base which specifically hybridizes thereto is used as a primer (PCR Experimental Protocol of Plants (Cell Technology, separate volume, Plant Cell Technology Series 2) Shujunsha Co. Ltd. (1995)).

Moreover, the DNA molecules encoding the aforementioned caffeine biosynthetic methyltransferases can be also isolated by performing hybridization screening of a cDNA library or a genomic library derived from an organism which produces the caffeine biosynthetic methyltransferase using the full length or partial DNA molecule thereof as a probe (Bio Experiments Illustrated 4, Cloning with Less Care (Cell Technology, separate volume, Visual Experimental Note Series) Shujunsha Co. Ltd. (1997)).

Such a DNA molecule encoding the caffeine biosynthetic methyltransferase obtained by the PCR technique or hybridization screening, or other method may have any nucleotide sequence as long as the translation product thereof has the intended methyltransferase activity, and may not necessarily has homology to any one of the sequence set out in SEQ ID NO: 2, 5 or 8.

Although the organism used for isolating the DNA molecules encoding the aforementioned caffeine biosynthetic methyltransferases may be any one as long as it produces methylxanthines, it is preferably a plant in genus coffee (Coffea), a plant in genus camellia (Camellia), a plant in genus cola (Cola), a plant in genus ilex (Ilex) and a plant in genus chocolate tree (Theobroma) for isolating a DNA molecule having the nucleotide sequence set out in SEQ ID NO: 2, 5 or 8, or a DNA molecule having homology to any one of these molecules.

The RNA molecules encoding the aforementioned caffeine biosynthetic methyltransferases can be obtained by ligating the caffeine biosynthetic methyltransferase DNA, which was obtained according to the aforementioned process, downstream of a promoter recognized by an RNA polymerase such as Sp6 RNA promoter or T7 promoter followed by transcription with Sp6 RNA polymerase, T7 RNA polymerase or the like. Alternatively, the RNA molecules can be also obtained through the use of the transcriptional activity of the host organism by forming a recombinant molecule through inserting a DNA or an RNA encoding the caffeine biosynthetic methyltransferase into an animal or plant virus or through inserting the caffeine biosynthetic methyltransferase DNA into an appropriate gene expression cassette as described hereinafter followed by introducing this recombinant molecule into the host organism.

For the expression and preparation of a recombinant protein from the DNA molecule or RNA molecule encoding the aforementioned caffeine biosynthetic methyltransferase, 1) a prokaryotic cellular expression system, 2) a yeast expression system, 3) a plant cellular expression system, 4) an insect cellular expression system, 5) a mammalian cellular expression system, 6) an in vitro transcription/translation system or the like can be utilized.

For obtaining the purified sample of the aforementioned recombinant protein, use of the prokaryotic cellular expression system (1) is most advantageous. For example, when GST Gene Fusion System (Amersham Biosciences) is used, the entire procedure starting from the construction of a vector to the expression and purification of the recombinant protein can be performed according to the attached instruction manual.

For identifying the function of the DNA molecule or RNA molecule encoding the aforementioned caffeine biosynthetic methyltransferase, a process is performed in which a recombinant protein (crude extract or purified product) is prepared by the aforementioned process, and an enzymatic reaction mixture containing the recombinant protein, a methyl group acceptor (xanthosine, 7-methylxanthine or theobromine) and a methyl group donor (S-adenosyl-L-methionine) is incubated followed by the TLC analysis or HPLC analysis of the product in this enzymatic reaction mixture (see, Examples herein).

Whether or not the biosynthetic pathway of caffeine can be reconstituted by way of the composite utilization of the DNA molecules or RNA molecules encoding the aforementioned caffeine biosynthetic methyltransferases can be determined by a procedure in which the recombinant proteins (crude extract or purified product) are prepared by the aforementioned process, and an enzymatic reaction mixture containing two or more recombinant proteins, a methyl group acceptor (xanthosine or 7-methylxanthine) and a methyl group donor (S-adenosyl-L-methionine) is incubated followed by the TLC analysis or HPLC analysis of the product in this enzymatic reaction mixture (see, Examples herein).

In the process for reconstituting the biosynthetic pathway of caffeine in a transformed organism system according to the invention, any organism species can be utilized as a host as long as it meets either requirement of: 1) biologically synthesizing xanthosine and having an enzymatic activity of ribose removal of 7-methylxanthosine; or 2) biologically synthesizing 7-methylxanthine.

Among the organisms which meet the above requirement, general plants are suitable as a host because they are comparably strong against toxicity of methylxanthines.

In connection with the process for reconstituting the biosynthetic pathway of caffeine in a transformed organism system according to the invention, examples in which a plant is used as a host is explained below.

For gene expression in a plant, any process can be utilized in which an expression cassette including 1) a promoter which enables transcription of from a DNA to an mRNA in host cells, 2) a caffeine biosynthetic methyltransferase DNA ligated downstream of the promoter in a sense direction, 3) a terminator sequence including a polyadenylation site required for stabilization of the transcription product ligated downstream of the DNA as needed, and the like is introduced into plant cells followed by transformation thereof.

The expression cassette may include a promoter for allowing the constitutive or inductive expression of the inserted DNA. Examples of the promoter for allowing the constitutive expression include 35S promoter of cauliflower mosaic virus, actin gene promoter of Oryza sativa, and the like. Examples of the promoter for allowing the inductive expression include chitinase gene promoter activated upon infection or invasion of a pathogenic microbe, PR protein gene promoter of tobacco, WIPK gene prompter of tobacco activated by lesion and the like.

Any of various techniques can be used for introducing the expression cassette into plant cells. Examples of these techniques include direct introduction methods (particle bombardment method, electroporation method and microinjection method), T-DNA introduction methods in which Agrobacterium tumefaciens is used as a transforming factor, and other possible techniques.

The direct introduction method does not require any specific vector. For example, simple plasmid such as a pUC derivative can be used. In the T-DNA introduction method, it is necessary to use a binary vector or the like containing a border sequence of T-DNA.

The transformed plant cell through introduction of the expression cassette can be converted into a plant tissue or a plant body via a regeneration process according to the conventional method.

For reconstituting the caffeine biosynthetic pathway, two or more types of expression cassettes must be introduced into plant cells.

When the direct introduction method is used, the transformant having a reconstituted biosynthetic pathway of caffeine can be obtained through the introduction of single vector containing multiple types of expression cassettes or multiple vectors containing single or multiple expression cassettes into plant cells. Alternatively, the transformant having a reconstituted biosynthetic pathway of caffeine can be also obtained through the introduction of single vector containing single or multiple expression cassettes into plant cells followed by mating of thus resulting transformant with other transformant.

When the T-DNA introduction method is used, the transformant having a reconstituted biosynthetic pathway of caffeine can be obtained through the introduction of single T-DNA containing multiple types of expression cassettes. Alternatively, the transformant having a reconstituted biosynthetic pathway of caffeine can be also obtained through the introduction of single T-DNA containing single or multiple expression cassettes into plant cells followed by mating of thus resulting transformant with other transformant.

A plant body (including tissue or cell) generated according to any of these methods or a plant body obtained from the propagation medium (such as seed, tuber or cut spike) has a reconstituted biosynthetic pathway of caffeine and produces at least one of the methylxanthines i.e., 7-methylxanthine, theobromine and caffeine.

The amount of production, the ratio of production and the like of methylxanthines in the plant having a reconstituted biosynthetic pathway of caffeine can be determined by the HPLC analysis. This HPLC analysis can be performed under a similar condition to those demonstrated in Examples herein.

Plants which produce theobromine or caffeine through the reconstitution of the biosynthetic pathway of caffeine have feeding-repellent effects and insecticidal effects against herbivore (insects, slug, snail and the like). In other words, such plant bodies endogenously have pesticidal effects.

### EXAMPLES

The present invention is specifically explained by way of the following examples. However, the scope of the invention is not limited to these examples.

(1) Synthesis of Double Stranded cDNA from mRNA Derived from Coffee Plants

Total RNA was extracted from 5 g of immature fruits of coffee plants (Coffea arabica) by a CTAB method (see, Chang et al., Plant Mol. Biol. Rep., 11, 113-116 (1993)). Using PolyATtract mRNA Isolation System III (Promega), mRNA was purified from 100 μg of the total RNA. Double stranded cDNA was synthesized using 5 μg of the mRNA and ZAP-cDNA Synthesis Kit (Stratagene).

(2) Isolation of Novel cDNA having Homology to CaMXMT cDNA and CaMTL3 cDNA

CaCS-N2 primer (5-ATGGAGCTCCAAGAAGTCCT-3) and CaCS-C1 primer (5-CTTTTACACGTCTGACTTCTCTG-3) were synthesized which were designed on the basis of a conserved sequence of CaMXMT cDNA (DDBJ/GenBank/EMBL accession number AB048794) and CaMTL3 cDNA (SEQ ID NO: 5, DDBJ/GenBank/EMBL accession number AB048793). PCR was carried out using the aforementioned cDNA, primers and Pyrobest DNA Polymerase (Takara Shuzo Co., Ltd.) to amplify a group of cDNA fragments. This group of cDNA fragments was inserted into EcoRV site of a vector pBluescript II KS-(Stratagene) to produce a plasmid library. Nucleotide sequence determination was conducted for plasmid clones randomly selected among the plasmid library, and CaMTL3 cDNA (SEQ ID NO: 2), and novel CaMTL4 cDNA (SEQ ID NO: 5) and CaMTL5 cDNA (SEQ ID NO: 8) having high homology thereto were isolated.

(3) Construction of GST Fusion Protein Expression Vector

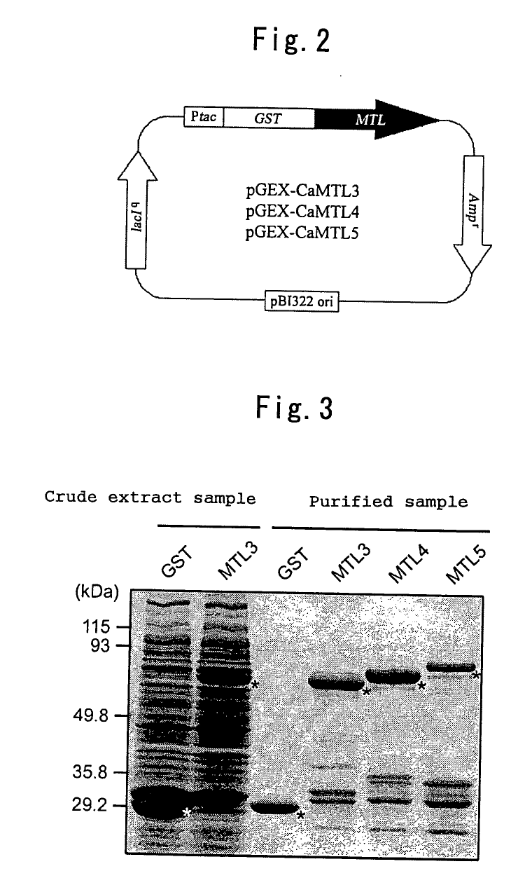

CaMTL3 cDNA, CaMTL4 cDNA and CaMTL5 cDNA were excised from the plasmid clones, and were inserted downstream of the glutathione S-transferase (GST) gene of pGEX-4T-2 vector (Amersham Biosciences), respectively. Through the procedure hereinabove, pGEX-CaMTL3 vector for allowing the expression of the fusion protein of GST and CaMTL3 (GST-MTL3), pGEX-CaMTL4 vector for allowing the expression of the fusion protein of GST and CaMTL4 (GST-MTL4), and pGEX-CaMTL5 vector for allowing the expression of the fusion protein of GST and CaMTL5 (GST-MTL5) were constructed. Thus resulting pGEX-CaMTL3 vector, pGEX-CaMTL4 vector and pGEX-CaMTL5 vector are schematically illustrated in FIG. 2. In FIG. 2, each vector was constructed by ligating CaMTL3 cDNA (SEQ ID NO: 2), CaMTL4 cDNA (SEQ ID NO: 5) or CaMTL5 cDNA (SEQ ID NO: 8), which is a predicted methyltransferase gene (MTL), downstream of a glutathione S-transferase gene (GST) controlled by tac promoter (Ptac).

(4) Production of Recombinant Protein Escherichia coli BL21 were transformed with vector pGEX-4T-2, pGEX-CaMTL3, pGEX-CaMTL4 and pGEX-CaMTL5. Any of the transformants was subjected to shaking culture in 100 mL of 2×YT liquid medium (supplemented with 100 mg/L ampicillin) at 37° C. until the absorbance (A600) of the culture becomes about 0.7. To these cultures was added isopropyl-β-D-thiogalactoside (IPTG) to give the final concentration of 1 mM followed by additional shaking culture at 20° C. for 16 hours. Through the procedure hereinabove, production of recombinant proteins (GST, GST-MTL3, GST-MTL4 and GST-MTL5) in Escherichia coli was executed.

(5) Crude Extract ion and Purification of Recombinant Protein

Escherichia coli including the produced recombinant protein were collected by centrifugation and suspended in 10 mL of a disintegrating wash fluid (50 mM Tris-HCl [pH 8.0], 1 mM EDTA, 5 mM dithiothreitol). The following procedure was carried out on ice or at 4° C. After thus resulting suspension was subjected to an ultrasonic disintegrating treatment, Triton X-100 was added thereto to give the final concentration of 1% followed by incubation for 30 minutes. Thus resulting disintegration fluid was separated to a supernatant and precipitates by centrifugation. This supernatant was referred to as a crude extract sample of the recombinant protein. To the crude extract sample was added a 100 μL glutathione Sepharose 4B resin (Amersham Biosciences), and the entire liquid was gently stirred for 30 minutes. The glutathione Sepharose 4B resin bound with the recombinant protein was recovered by centrifugation and washed three times with 1 mL of the disintegrating wash fluid. Thereafter, 100 μL of a lysis solution (50 mM Tris-HCl [pH8.5], 1 mM EDTA, 5mM dithiothreitol, 10 mM glutathione) was added to the resin, and the mixture was gently stirred for 15 minutes. From the mixture was recovered the eluate by centrifugation, which was referred to as a purified sample of the recombinant protein. SDS-PAGE analysis verified that the recombinant protein was included in the crude extract sample and the purified sample. Results of detection of the recombinant protein by the SDS-PAGE analysis are shown in FIG. 3.

(6) Identification of Enzymatic Reaction Product by Thin Layer Chromatography (TLC)

Following enzymatic reaction experiments and TLC analyses were performed for the purified GST, crude extract GST, purified GST-MTL3, crude extract GST-MTL3, purified GST-MTL4 and purified GST-MTL5. An enzyme-containing mixture in an amount of 25 μL which comprises 100 mM Tris-HCl [pH 8.0], 200 μM MgCl₂, 500 μM substrate (xanthosine [XR], 7-methylxanthine [7mX] or theobromine [Tb]), 16.8 μM S-adenosyl-L-[methyl-¹⁴C]methionine [SAM] [2.2 GBq/mmol; Amersham Biosciences] and the recombinant protein sample (50 μg of the crude extract sample or 5 μg of the purified sample) was incubated at 27° C. for 16 hours.

The enzymatic reaction mixture after the incubation was filtered with a filter cup (ULTRAFREE-MC 10,000 NMWL; Millipore) The filtrate in an amount of 2 μL was mixed with 2 μL methanol, and the resulting mixture was spotted on a TLC plate (Silicagel 60 F254; Merck).

Alternatively, the reaction product was purified by adding 250 μL of chloroform to the enzymatic reaction mixture after the incubation, vigorously stirring the entire solution, recovering the chloroform layer, which was subjected to concentration and evaporation to dryness. This purified product was suspended in 4 μL of 50% methanol, and spotted on a TLC plate.

TLC analyses were performed similarly to those as described above using any one of 7-methylxanthosine [7mXR], [7mX], [Tb], caffeine [Cf] and S-adenosyl-L-methionine [SAM] (1 mM aqueous solution, each) as a standard, instead of the enzymatic reaction mixture and the purified product. A mixture of 4 μL of the standard and 4 μL of methanol was spotted on a TLC plate.

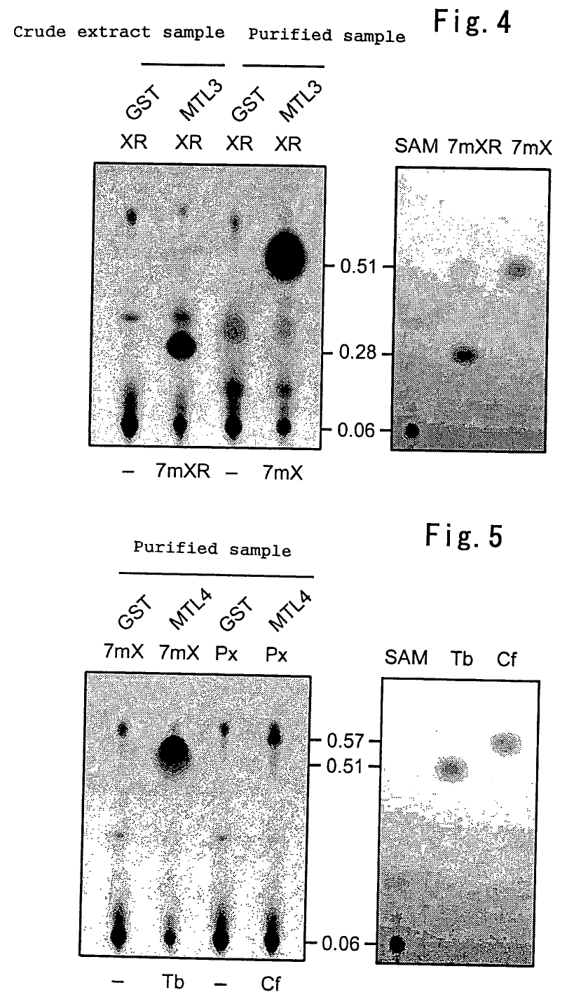

Thereafter, development was conducted using water/acetic acid/n-butanol (2:1:4, v/v/v) as a developing solvent for 1.5 hrs. A sensitizing agent (En³ Hance; Dupont NEN) was sprayed onto the part with the developed reaction product present. Autoradiography was performed using an X-ray film (BioMax MS; Kodak) through the exposure at −80° C. for 3 days to detect the spot of the enzymatic reaction product having a ¹⁴C-labelled methyl group added. The spot of the standard was detected by illuminating UV light onto the part with the developed standard. Results of analyses by TLC are illustrated in FIG. 4 to FIG. 6.

As is clear from FIG. 4, the purified sample of GST-MTL3 produced [7mXR] in the presence of [XR] which is a methyl group acceptor and [SAM] which is a methyl group donor. In other words, it was revealed that this sample catalyzed a productive reaction of [7mXR] through methylation of [XR].

Further, the crude extract sample of GST-MTL3 produced [7mX] which is a ribose moiety-removed product of [7mXR] in the presence of [XR] which is a methyl group acceptor and [SAM] which is a methyl group donor. In other words, it was revealed that this sample catalyzed two reactions of producing [7mXR] through methylation of [XR] and producing [7mX] through ribose removal of [7mXR].

As is clear from FIG. 5, the purified sample of GST-MTL4 produced [Tb] in the presence of [7mX] which is a methyl group acceptor and [SAM] which is a methyl group donor. In other words, it was revealed that this sample catalyzed a productive reaction of [Tb] through methylation of [7mX].

Further, the purified sample of GST-MTL4 produced [Cf] in the presence of paraxanthine [Px] which is a methyl group acceptor and [SAM] which is a methyl group donor. In other words, it was revealed that this sample catalyzed a productive reaction of [Cf] through methylation of [Px].

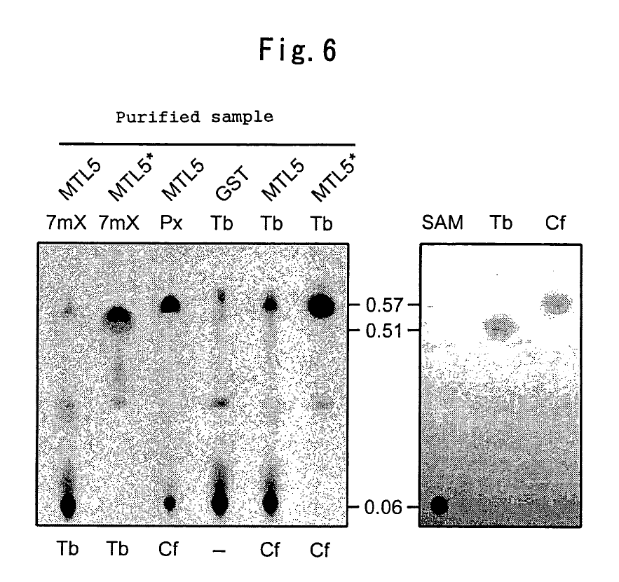

As is clear from FIG. 6, the purified sample of GST-MTL5 produced [Cf] in the presence of [Tb] which is a methyl group acceptor and [SAM] which is a methyl group donor. In other words, it was revealed that this sample catalyzed a productive reaction of [Cf] through methylation of [Tb].

Further, the purified sample of GST-MTL5 produced [Tb] in the presence of [7mX] which is a methyl group acceptor and [SAM] which is a methyl group donor. In other words, it was revealed that this sample catalyzed a productive reaction of [Tb] through methylation of [7mX].

Moreover, the purified sample of GST-MTL5 produced [Cf] in the presence of [Px] which is a methyl group acceptor and [SAM] which is a methyl group donor. In other words, it was revealed that this sample catalyzed a productive reaction of [Cf] through methylation of [Px].

GST which was used as a control did not catalyze any methylation reaction in both of the purified sample and the crude extract sample.

Accordingly, it was revealed that the CaMTL3 protein, CaMTL4 protein and CaMTL5 protein, and a group of genes encoding these proteins are constituent elements of the biosynthetic pathway of caffeine, respectively. (7) Reconstitution of Biosynthetic Pathway of Caffeine in vitro

An enzyme-containing mixture in an amount of 100 μL which comprises 100 mM Tris-HCl [pH8.0], 200 μM MgCl₂, 500 μM xanthosine [XR], 1.5 mM S-adenosyl-L-methionine [SAM] and the recombinant protein sample (200 μg of the crude extract sample or 20 μg of the purified sample) was incubated at 27° C. for 16 hours. The aforementioned recombinant protein samples used in the enzymatic reaction were: (A) crude extract GST-MTL3 alone, (B) combination of crude extract GST-MTL3 and purified GST-MTL4, and (C) combination of crude extract GST-MTL3, purified GST-MTL4 and purified GST-MTL5.

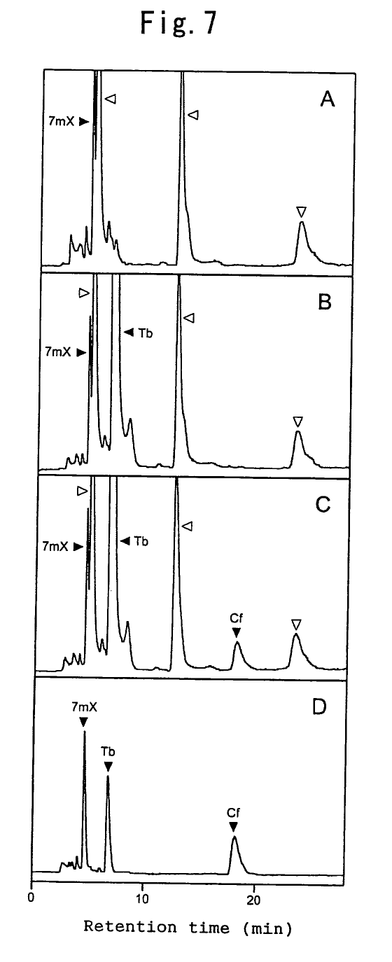

The reaction product was purified by adding 1 mL of chloroform to the enzymatic reaction mixture after the incubation, vigorously stirring the entire solution, recovering the chloroform layer, which was subjected to concentration and evaporation to dryness. This purified product was suspended in 200 μL of an HPLC developing solvent (50 mM sodium phosphate [pH 6.0]/methanol [4:1, v/v]) and 20 μL of the suspension was used for the HPLC analysis. Puresil C18 (Waters) was used as a column for the HPLC analysis, and the suspension was developed at a flow rate of 1 mL/min. The enzymatic reaction product was detected by absorption of UV light(270 nm). HPLC analyses were performed similarly to those as described above using any one of 7-methylxanthine [7mX], theobromine [Tb] and caffeine [Cf] as a standard, instead of the enzymatic reaction product. Results of analyses by HPLC are illustrated in FIG. 7.

As is clear from FIG. 7-A, the crude extract sample of GST-MTL3 produced [7mX] which is a ribose moiety-removed product of [7mXR] in the presence of [XR] which is a methyl group acceptor and [SAM] which is a methyl group donor. In other words, it was determined that this sample catalyzed two reactions of producing [7mXR] through methylation of [XR] and producing [7mX] through ribose removal of [7mXR] in the HPLC analysis as well similarly to the TLC analysis.

As is clear from FIG. 7-B, a combination of the crude extract sample of GST-MTL3 and the purified sample of GST-MTL4 produced [7mX] and [Tb] in the presence of [XR] and [SAM]. In other words, it was revealed that the combination of these samples catalyzed three reactions of producing [7mXR] through methylation of [XR], producing [7mX] through ribose removal of [7mXR], and producing [Tb] through methylation of [7mX].

As is clear from FIG. 7-C, a combination of the crude extract sample of GST-MTL3, the purified sample of GST-MTL4 and the purified sample of GST-MTL5 produced [7mX], [Tb] and [Cf] in the presence of [XR] and [SAM]. In other words, it was revealed that the combination of these samples catalyzed four reactions of producing [7mXR] through methylation of [XR], producing [7mX] through ribose removal of [7mXR], producing [Tb] through methylation of [7mX], and producing [Cf] through methylation of [Tb].

As in the foregoing, it was proven that a biosynthetic pathway of caffeine can be reconstituted in vitro through composite utilization of the CaMTL3 protein, CaMTL4 protein, CaMTL5 protein and crude extract of Escherichia coli.

In accordance with the present invention, effects as described below are exerted.

(1) Using two or more of the multiple enzymes in combination which respectively catalyze each step in a series of the caffeine biosynthetic reaction system, 7-methylxanthine, theobromine or caffeine can be produced ex vivo through executing methylation of xanthosine at the 7-position of the purine ring, ribose removal of 7-methylxanthosine at the 9-position of the purine ring, methylation of 7-methylxanthine at the 3-position of the purine ring, and/or methylation of theobromine at the 1-position of the purine ring.

(2) Using two or more of the multiple genes in the biosynthetic pathway of caffeine in combination which encode multiple enzymes which respectively catalyze each step in a series of the biosynthetic reaction system of caffeine, the amount of production of theobromine or caffeine can be altered through allowing the expression of this combination in a host organism which biologically synthesizes theobromine or caffeine.

(3) Using two or more of the multiple genes in the biosynthetic pathway of caffeine in combination which encode multiple enzymes which respectively catalyze each step in a series of the biosynthetic reaction system of caffeine, theobromine or caffeine can be produced through allowing the expression of this combination in a host organism which biologically synthesizes 7-methylxanthine to alter the metabolism of the host organism.

(4) Using two or more of the multiple genes in the biosynthetic pathway of caffeine in combination which encode multiple enzymes which respectively catalyze each step in a series of the biosynthetic reaction system of caffeine, theobromine or caffeine can be produced through allowing the expression of the combination in a host organism which biologically synthesizes xanthosine and has an enzymatic activity of ribose removal of 7-methylxanthosine to alter the metabolism of the host organism.

(5) According to the process of the above (3) or (4), metabolism of the organism which does not biologically synthesize theobromine or caffeine in itself is altered to allow the organism to produce these compounds.

(6) Through performing the aforementioned process (3) or (4) in a plant, it becomes possible to defend the plant from pest feeding by a herbivore.

What is claimed is:

1. A process for producing 7-methylxanthine, theobromine or caffeine which comprises: methylation of xanthosine at the 7-position of the purine ring; ribose removal of 7-methylxanthosine at the 9-position of the purine ring; methylation of 7-methylxanthine at the 3-position of the purine ring; and/or methylation of theobromine at the 1-position of the purine ring ex vivo, under the catalytic action of a combination of two or more of the following enzymes (a), (c) and (d), and the cellular extract (b):
(a) an enzyme having a catalytic activity of methylation of xanthosine at the 7-position of the purine ring and having the amino acid sequence set out in SEQ ID NO: 1,
(b) a crude cellular extract obtained from Escherichia coli having a catalytic activity of ribose removal of 7-methylxanthosine at the 9-position of the purine ring,
(c) an enzyme having a catalytic activity of methylation of 7-methylxanthine at the 3-position of the purine ring and having the amino acid sequence set out in SEQ ID NO: 4,
(d) an enzyme having a catalytic activity of methylation of theobromine at the 1-position of the purine ring and having the amino acid sequence set out in SEQ ID NO: 7.

2. The process according to claim 1 wherein at least one of the enzymes (a), (c) and (d), and the cellular extract (b) is substituted for one having a comparable activity thereto.

3. A process for producing theobromine or caffeine which comprises allowing the expression of a combination of (a)+(b) or a combination of (a)+(b)+(c) of the following DNA molecules (a), (b) and (c) in a host organism which biologically synthesizes xanthosine and has an enzymatic activity of ribose removal of 7-methylxanthosine to alter the metabolism of the host organism:
(a) a DNA molecule encoding an enzyme which catalyzes methylation of xanthosine at the 7-position of the purine ring and having the nucleotide sequence set out in SEQ ID NO: 2,
(b) a DNA molecule encoding an enzyme which catalyzes methylation of 7-methylxanthine at the 3-position of the purine ring and having the nucleotide sequence set out in SEQ ID NO: 5,
(c) a DNA molecule encoding an enzyme which catalyzes methylation of theobromine at the 1-position of the purine ring and having the nucleotide sequence set out in SEQ ID NO: 8.

4. A process for producing theobromine or caffeine which comprises allowing the expression of a combination of the following DNA molecules (b) and (c) in a host organism which biologically synthesizes 7-methylxanthine to alter the metabolism of the host organism:
(b) a DNA molecule encoding an enzyme which catalyzes methylation of 7-methylxanthine at the 3-position of the purine ring and having the nucleotide sequence set out in SEQ ID NO: 5,
(c) a DNA molecule encoding an enzyme which catalyzes methylation of theobromine at the 1-position of the purine ring and having the nucleotide sequence set out in SEQ ID NO: 8.

5. A process for altering the amount of production of theobromine or caffeine which comprises allowing the expression of a combination of two or more of the following DNA molecules (a), (b) and (c) in a host organism which biologically synthesizes theobromine or caffeine:
(a) a DNA molecule encoding an enzyme which catalyzes methylation of xanthosine at the 7-position of the purine ring and having the nucleotide sequence set out in SEQ ID NO:2,
(b) a DNA molecule encoding an enzyme which catalyzes methylation of 7-methylxanthine at the 3-position of the purine ring and having the nucleotide sequence set out in SEQ ID NO: 5,
(c) a DNA molecule encoding an enzyme which catalyzes methylation of theobromine at the 1-position of the purine ring and having the nucleotide sequence set out in SEQ ID NO: 8.

6. The process according to any one of claims 3 to 5 wherein at least one of the DNA molecules (a), (b) and (c) is substituted for one having a comparable function thereto.

7. A process for producing theobromine or caffeine which comprises allowing the expression of a combination of (a)+(b) or a combination of (a)+(b)+(c) of the following RNA molecules (a), (b) and (c) in a host organism which biologically synthesizes xanthosine and has an enzymatic activity of ribose removal of 7-methylxanthosine to alter the metabolism of the host organism:
(a) an RNA molecule encoding an enzyme which catalyzes methylation of xanthosine at the 7-position of the purine ring and having the nucleotide sequence set out in SEQ ID NO: 3,
(b) an RNA molecule encoding an enzyme which catalyzes methylation of 7-methylxanthine at the 3-position of the purine ring and having the nucleotide sequence set out in SEQ ID NO: 6,
(c) an RNA molecule encoding an enzyme which catalyzes methylation of theobromine at the 1-position of the purine ring and having the nucleotide sequence set out in SEQ ID NO: 9.

8. A process for producing theobromine or caffeine which comprises allowing the expression of a combination of the following RNA molecules (b) and (c) in a host organism which biologically synthesizes 7-methylxanthine to alter the metabolism of the host organism:
(b) an RNA molecule encoding an enzyme which catalyzes methylation of 7-methylxanthine at the 3-position of the purine ring and having the nucleotide sequence set out in SEQ ID NO: 6,
(c) an RNA molecule encoding an enzyme which catalyzes methylation of theobromine at the 1-position of the purine ring and having the nucleotide sequence set out in SEQ ID NO: 9.

9. A process for altering the amount of production of theobromine or caffeine which comprises allowing the expression of a combination of two or more of the following RNA molecules (a), (b) and (c) in a host organism which biologically synthesizes theobromine or caffeine:
(a) an RNA molecule encoding an enzyme which catalyzes methylation of xanthosine at the 7-position of the purine ring and having the nucleotide sequence set out in SEQ ID NO:3,
(b) an RNA molecule encoding an enzyme which catalyzes methylation of 7-methylxanthine at the 3-position of the purine ring and having the nucleotide sequence set out in SEQ ID NO: 6,
(c) an RNA molecule encoding an enzyme which catalyzes methylation of theobromine at the 1-position of the purine ring and having the nucleotide sequence set out in SEQ ID NO: 9.

10. The process according to any one of claims 7 to 9 wherein at least one of the RNA molecules (a), (b) and (c) is substituted for one having a comparable function thereto.

11. The process according to any one of claims 3 to 10 wherein the host organism is a plant, and production or increase of production of theobromine or caffeine defends the host plant from pest feeding by a herbivore.


SEQUENCE LISTING (11 sequences)

SEQ ID NO: 1 - protein, 372 aa, Coffee Arabica
  ref <301>: AUTHORS: Ogawa et al.
  ref <302>: TITLE: 7-Methylxanthine Methyltransferase of Coffee Plants, Gene Isolation and Enzymatic Properties
  ref <303>: JOURNAL: Journal of Biological Chemistry
  ref <304>: VOLUME: 276
  ref <305>: ISSUE: 11
  ref <306>: PAGES: 8213-8218
  ref <307>: DATE: 2001-03-16
  ref <308>: DATABASE ACCESSION NUMBER: BAB39215
  ref <309>: DATABASE ENTRY DATE: 2000-09-11
MELQEVLRMNGGEGDTSYAKNSAYNQLVLAKVKPVLEQCVRELLRANLPNINKCIKVADLGCASGPNTLLTVRDIVQSIDKVGQEKKNELERPTIQIFLNDLFPNDFNSVFKLLPSFYRKLEKENGRKIGSCLIGAMPGSFYSRLFPEESMHFLHSCYCLQWLSQVPSGLVTELGISTNKGSIYSSKASRLPVQKAYLDQFTKDFTTFLRIHSEELFSHGRMLLTCICKGVELDARNAIDLLEMAINDLVVEGHLEEEKLDSFNLPVYIPSAEEVKCIVEEEGSFEILYLETFKVLYDAGFSIDDEHIKAEYVASSVRAVYEPILASHFGEAIIPDIFHRFAKHAAKVLPLGKGFYNNLIISLAKKPEKSDV

SEQ ID NO: 2 - DNA, 1316 bp, Coffee Arabica
  CDS (45)..(1163)
  ref <308>: DATABASE ACCESSION NUMBER: AB048793
  ref <309>: DATABASE ENTRY DATE: 2000-09-11
ctttggcagtcccaatttgatttatgtacaagtcctgcatatgaatggagctccaagaagtcctgcggatgaatggaggcgaaggcgatacaagctacgccaagaattcagcctacaatcaactggttctcgccaaggtgaaacctgtccttgaacaatgcgtacgggaattgttgcgggccaacttgcccaacatcaacaagtgcattaaagttgcggatttgggatgcgcttctggaccaaacacacttttaacagttcgggacattgtccaaagtattgacaaagttggccaggaaaagaagaatgaattagaacgtcccaccattcagatttttctgaatgatcttttcccaaatgatttcaattcggttttcaagttgctgccaagcttctaccgcaaacttgagaaagaaaatggacgcaaaataggatcgtgcctaataggggcaatgcccggctctttctacagcagactcttccccgaggagtccatgcattttttacactcttgttactgtcttcaatggttatctcaggttcctagcggtttggtgactgaattggggatcagtacgaacaaagggagcatttactcttccaaagcaagtcgtctgcccgtccagaaggcatatttggatcaatttacgaaagattttaccacatttctaaggattcattcggaagagttgttttcacatggccgaatgctccttacttgcatttgtaaaggagttgaattagacgcccggaatgccatagacttacttgagatggcaataaacgacttggttgttgagggacatctggaggaagaaaaattggatagtttcaatcttccagtctatataccttcagcagaagaagtaaagtgcatagttgaggaggaaggttcttttgaaattttatacctggagacttttaaggtcctttacgatgctggcttctctattgacgatgaacatattaaagcagagtatgttgcatcttccgttagagcagtttacgaacccatcctcgcaagtcattttggagaagctattatacctgacatattccacaggtttgcgaagcatgcagcaaaggttctccccttgggcaaaggcttctataataatcttatcatttctctcgccaaaaagccagagaagtcagacgtgtaaaagtttgtttttgtgttggggaaaggaataagtgccgttgggggtctttcgggtattgtgctttttatattatattgttttgtatccgtaataaaagtggtgtgtaagaataagatatttgacatatattattttcaaaaaaaaaaaaaaaaa

SEQ ID NO: 3 - RNA, 1316 nt, Coffee Arabica
  CDS (45)..(1163)
  ref <308>: DATABASE ACCESSION NUMBER: AB048793
  ref <309>: DATABASE ENTRY DATE: 2000-09-11
cuuuggcagucccaauuugauuuauguacaaguccugcauaugaauggagcuccaagaaguccugcggaugaauggaggcgaaggcgauacaagcuacgccaagaauucagccuacaaucaacugguucucgccaaggugaaaccuguccuugaacaaugcguacgggaauuguugcgggccaacuugcccaacaucaacaagugcauuaaaguugcggauuugggaugcgcuucuggaccaaacacacuuuuaacaguucgggacauuguccaaaguauugacaaaguuggccaggaaaagaagaaugaauuagaacgucccaccauucagauuuuucugaaugaucuuuucccaaaugauuucaauucgguuuucaaguugcugccaagcuucuaccgcaaacuugagaaagaaaauggacgcaaaauaggaucgugccuaauaggggcaaugcccggcucuuucuacagcagacucuuccccgaggaguccaugcauuuuuuacacucuuguuacugucuucaaugguuaucucagguuccuagcgguuuggugacugaauuggggaucaguacgaacaaagggagcauuuacucuuccaaagcaagucgucugcccguccagaaggcauauuuggaucaauuuacgaaagauuuuaccacauuucuaaggauucauucggaagaguuguuuucacauggccgaaugcuccuuacuugcauuuguaaaggaguugaauuagacgcccggaaugccauagacuuacuugagauggcaauaaacgacuugguuguugagggacaucuggaggaagaaaaauuggauaguuucaaucuuccagucuauauaccuucagcagaagaaguaaagugcauaguugaggaggaagguucuuuugaaauuuuauaccuggagacuuuuaagguccuuuacgaugcuggcuucucuauugacgaugaacauauuaaagcagaguauguugcaucuuccguuagagcaguuuacgaacccauccucgcaagucauuuuggagaagcuauuauaccugacauauuccacagguuugcgaagcaugcagcaaagguucuccccuugggcaaaggcuucuauaauaaucuuaucauuucucucgccaaaaagccagagaagucagacguguaaaaguuuguuuuuguguuggggaaaggaauaagugccguugggggucuuucggguauugugcuuuuuauauuauauuguuuuguauccguaauaaaagugguguguaagaauaagauauuugacauauauuauuuucaaaaaaaaaaaaaaaaa

SEQ ID NO: 4 - protein, 384 aa, Coffee Arabica
MELQEVLHMNEGEGDTSYAKNASYNLALAKVKPFLEQCIRELLRANLPNINKCIKVADLGCASGPNTLLTVRDIVQSIDKVGQEEKNELERPTIQIFLNDLFQNDFNSVFKLLPSFYRKLEKENGRKIGSCLISAMPGSFYGRLFPEESMHFLHSCYSVHWLSQVPSGLVIELGIGANKGSIYSSKASRPPVQKAYLDQFTKDFTTFLRIHSKELFSRGRMLLTCICKVDEYDEPNPLDLLDMAINDLIVEGHLEEEKLASFNLPFFTPSAEEVKCIVEEEGSFEILYLETFKAHYDAGFSIDDDYPVRSHFQVYGDEHIKAEYVASLIRSVYEPILASHFGEAIMPDLFHRLAKHAAKVLHLGKGCYNNLIISLAKKPEKSDV

SEQ ID NO: 5 - DNA, 1155 bp, Coffee
  CDS (1)..(1152)
atggagctccaagaagtcctgcatatgaatgaaggtgaaggcgatacaagctacgccaagaatgcatcctacaatctggctcttgccaaggtgaaacctttccttgaacaatgcatacgagaattgttgcgggccaacttgcccaacatcaacaagtgcattaaagttgcggatttgggatgcgcttctggaccaaacacacttttaacagtgcgggacattgtgcaaagtattgacaaagttggccaggaagagaagaatgaattagaacgtcccaccattcagatttttctgaatgatcttttccaaaatgatttcaattcggttttcaagttgctgccaagcttctaccgcaaactcgagaaagaaaatggacgcaagataggatcgtgcctaataagcgcaatgcctggctctttctacggcagactcttccccgaggagtccatgcattttttgcactcttgttacagtgttcattggttatctcaggttcccagcggtttggtgattgaattggggattggtgcaaacaaagggagtatttactcttccaaagcaagtcgtccgcccgtccagaaggcatatttggatcaatttacgaaagattttaccacatttctaaggattcattcgaaagagttgttttcacgtggccgaatgctccttacttgcatttgtaaagtagatgaatacgacgaaccgaatcccctagacttacttgacatggcaataaacgacttgattgttgagggacatctggaggaagaaaaattggctagtttcaatcttccattctttacaccttcagcagaagaagtaaagtgcatagttgaggaggaaggttcttttgaaattttatacctggagacttttaaggcccattatgatgctggcttctctattgatgatgattacccagtaagatcccatttccaagtatacggcgatgaacatattaaagcagagtatgtggcatcattaattagatcagtttacgaacccatcctcgcaagtcattttggagaagctattatgcctgacttattccacaggcttgcgaagcatgcagcaaaggttctccacttgggcaaaggctgctataataatcttatcatttctctcgccaaaaagccagagaagtcagacgtgtaa

SEQ ID NO: 6 - RNA, 1155 nt, Coffee Arabica
  CDS (1)..(1152)
auggagcuccaagaaguccugcauaugaaugaaggugaaggcgauacaagcuacgccaagaaugcauccuacaaucuggcucuugccaaggugaaaccuuuccuugaacaaugcauacgagaauuguugcgggccaacuugcccaacaucaacaagugcauuaaaguugcggauuugggaugcgcuucuggaccaaacacacuuuuaacagugcgggacauugugcaaaguauugacaaaguuggccaggaagagaagaaugaauuagaacgucccaccauucagauuuuucugaaugaucuuuuccaaaaugauuucaauucgguuuucaaguugcugccaagcuucuaccgcaaacucgagaaagaaaauggacgcaagauaggaucgugccuaauaagcgcaaugccuggcucuuucuacggcagacucuuccccgaggaguccaugcauuuuuugcacucuuguuacaguguucauugguuaucucagguucccagcgguuuggugauugaauuggggauuggugcaaacaaagggaguauuuacucuuccaaagcaagucguccgcccguccagaaggcauauuuggaucaauuuacgaaagauuuuaccacauuucuaaggauucauucgaaagaguuguuuucacguggccgaaugcuccuuacuugcauuuguaaaguagaugaauacgacgaaccgaauccccuagacuuacuugacauggcaauaaacgacuugauuguugagggacaucuggaggaagaaaaauuggcuaguuucaaucuuccauucuuuacaccuucagcagaagaaguaaagugcauaguugaggaggaagguucuuuugaaauuuuauaccuggagacuuuuaaggcccauuaugaugcuggcuucucuauugaugaugauuacccaguaagaucccauuuccaaguauacggcgaugaacauauuaaagcagaguauguggcaucauuaauuagaucaguuuacgaacccauccucgcaagucauuuuggagaagcuauuaugccugacuuauuccacaggcuugcgaagcaugcagcaaagguucuccacuugggcaaaggcugcuauaauaaucuuaucauuucucucgccaaaaagccagagaagucagacguguaa

SEQ ID NO: 7 - protein, 384 aa, Coffee Arabica
MELQEVLHMNGGEGDTSYAKNSFYNLFLIRVKPILEQCIQELLRANLPNINKCIKVADLGCASGPNTLLTVRDIVQSIDKVGQEKKNELERPTIQIFLNDLFQNDFNSVFKSLPSFYRKLEKENGRKIGSCLIGAMPGSFYGRLFPEESMHFLHSCYCLHWLSQVPSGLVTELGISANKGCIYSSKASRPPIQKAYLDQFTKDFTTFLRIHSEELISRGRMLLTWICKEDEFENPNSIDLLEMSINDLVIEGHLEEEKLDSFNVPIYAPSTEEVKCIVEEEGSFEILYLETFKVPYDAGFSIDDDYQGRSHSPVSCDEHARAAHVASVVRSIFEPIVASHFGEAIMPDLSHRIAKNAAKVLRSGKGFYDSLIISLAKKPEKSDV

SEQ ID NO: 8 - DNA, 1155 bp, Coffee Arabica
  CDS (1)..(1152)
atggagctccaagaagtcctgcatatgaatggaggcgaaggcgatacaagctacgccaagaactcattctacaatctgtttctcatcagggtgaaacctatccttgaacaatgcatacaagaattgttgcgggccaacttgcccaacatcaacaagtgcattaaagttgcggatttgggatgcgcttctggaccaaacacacttttaacagttcgggacattgtacaaagtattgacaaagttggccaggaaaagaagaatgaattagaacgtcccaccattcagatttttctgaatgatcttttccaaaatgatttcaattcggttttcaagtcgctgccaagcttctaccgcaaacttgagaaagaaaatggacgcaaaataggatcatgcctgataggcgcaatgcctggctctttctacggcagactcttccccgaggagtccatgcattttttacactcttgttactgtttgcattggttatctcaggttcccagcggtttggtgactgaattggggatcagtgcgaacaaagggtgcatttactcttccaaagcaagtcgtccgcccatccagaaggcatatttggatcaatttacgaaagattttaccacatttcttaggattcattcggaagagttgatttcacgtggccgaatgctccttacttggatttgcaaagaagatgaattcgagaacccgaattccatagacttacttgagatgtcaataaacgacttggttattgagggacatctggaggaagaaaaattggacagtttcaatgttccaatctatgcaccttcaacagaagaagtaaagtgcatagttgaggaggaaggttcttttgaaattttatacctggagacttttaaggtcccttatgatgctggcttctctattgatgatgattaccaaggaagatcccattccccagtatcctgcgatgaacatgctagagcagcgcatgtggcatctgtcgttagatcaattttcgaacccatcgtcgcaagtcattttggagaagctatcatgcctgacttatcccacaggattgcgaagaatgcagcaaaggttcttcgctccggcaaaggcttctatgatagtcttatcatttctctcgccaaaaagccagagaagtcagacgtgtaa

SEQ ID NO: 9 - RNA, 1155 nt, Coffee Arabica
  CDS (1)..(1152)
auggagcuccaagaaguccugcauaugaauggaggcgaaggcgauacaagcuacgccaagaacucauucuacaaucuguuucucaucagggugaaaccuauccuugaacaaugcauacaagaauuguugcgggccaacuugcccaacaucaacaagugcauuaaaguugcggauuugggaugcgcuucuggaccaaacacacuuuuaacaguucgggacauuguacaaaguauugacaaaguuggccaggaaaagaagaaugaauuagaacgucccaccauucagauuuuucugaaugaucuuuuccaaaaugauuucaauucgguuuucaagucgcugccaagcuucuaccgcaaacuugagaaagaaaauggacgcaaaauaggaucaugccugauaggcgcaaugccuggcucuuucuacggcagacucuuccccgaggaguccaugcauuuuuuacacucuuguuacuguuugcauugguuaucucagguucccagcgguuuggugacugaauuggggaucagugcgaacaaagggugcauuuacucuuccaaagcaagucguccgcccauccagaaggcauauuuggaucaauuuacgaaagauuuuaccacauuucuuaggauucauucggaagaguugauuucacguggccgaaugcuccuuacuuggauuugcaaagaagaugaauucgagaacccgaauuccauagacuuacuugagaugucaauaaacgacuugguuauugagggacaucuggaggaagaaaaauuggacaguuucaauguuccaaucuaugcaccuucaacagaagaaguaaagugcauaguugaggaggaagguucuuuugaaauuuuauaccuggagacuuuuaaggucccuuaugaugcuggcuucucuauugaugaugauuaccaaggaagaucccauuccccaguauccugcgaugaacaugcuagagcagcgcauguggcaucugucguuagaucaauuuucgaacccaucgucgcaagucauuuuggagaagcuaucaugccugacuuaucccacaggauugcgaagaaugcagcaaagguucuucgcuccggcaaaggcuucuaugauagucuuaucauuucucucgccaaaaagccagagaagucagacguguaa

SEQ ID NO: 10 - DNA, 20 bp, Artificial
  DNA Primer
atggagctccaagaagtcct

SEQ ID NO: 11 - DNA, 23 bp, Artificial
  DNA Primer
cttttacacgtctgacttctctg